(19) United States
(12) Patent Application Publication
(10) Pub. No.: US 2005/0013856 A1
(43) Pub. Date: Jan. 20, 2005

(54) Solid dispersions comprising a hygroscopic and/or deliquescent drug

(76) Inventors: Jay S. Trivedi, Skokie, IL (US); Rajeev D. Gokhale, Waukegan, IL (US)

Correspondence Address: PHARMACIA CORPORATION;GLOBAL PATENT DEPARTMENT, POST OFFICE BOX 1027, ST. LOUIS, MO, 63006, US

(21) Appl. No.: 10/741,526

(22) Filed: Dec. 18, 2003

Related U.S. Application Data
(60) Provisional application No. 60/435,147, filed on Dec. 19, 2002.
(60) Provisional application No. 60/435,022, filed on Dec. 19, 2002.
(60) Provisional application No. 60/435,422, filed on Dec. 19, 2002.

(51) Int. Cl. A61K 9/20; A61K 9/14

(52) U.S. Cl. 424/464; 424/488

(57) ABSTRACT

A pharmaceutical composition is provided comprising a drug and a carrier medium, wherein the carrier medium comprisese (a) a matrix forming agent selected from the group consisting of hydroxyethylcelluloses, hydroxypropylcelluloses, hydroxypropylmethylcelluloses, hydroxypropylmethylcellulose phthalates, polyvinylpyrrolidones, polyethylene glycols, polyglycolized glycerides, cyclodextrins, carbomers and combinations thereof, and (b) a filler; and wherein the drug is hygroscopic and/or deliquescent and is dispersed in the carrier medium, and wherein the composition is a solid dispersion and is acceptably non-hygroscopic.

This application claims priority of U.S. Provisional Application Ser. No. 60/435147 filed on 19 Dec. 2002, of U.S. Provisional Application Ser. No. 60/435022 filed on 19 Dec. 2002, and U.S. Provisional Application Ser. No. 60/435422 filed on 19 Dec. 2002.

### FIELD OF THE INVENTION

The present invention relates to acceptably non-hygroscopic pharmaceutical compositions that comprise a hygroscopic and/or deliquescent drug, more particularly to such compositions wherein the drug is incorporated within a solid dispersion.

### BACKGROUND OF THE INVENTION

The sorption of moisture by drugs can create significant problems. In the presence of moisture, a solid drug substance can become hydrated and/or convert to a new crystal form. Moisture sorption also can adversely affect release rate of the substance from a formulation, shelf life of a formulation, and handling and processing properties of the substance. Hygroscopic and/or deliquescent drugs, by definition, are prone to experiencing these adverse effects when exposed to environments with even moderate humidity. Thus, it is usually imperative to control moisture sorption during formulation development and storage.

In the past, alterations to the manufacturing plant, such as installation of machinery to lower humidity within the plant, have been used to limit exposure of hygroscopic and/or deliquescent drugs to humid conditions during their production. However, such alterations are disadvantageous in that they are costly, and sometimes unreliable in maintaining proper ambient conditions. Further, alterations in manufacturing conditions do very little in protecting a hygroscopic and/or deliquescent drug in humid storage conditions.

U.S. Pat. No. 5,225,204 to Chen et al., incorporated herein by reference, describes means to provide compositions of the hygroscopic drug levothyroxine sodium that are said to be stable in humid conditions. One such means consists of mixing levothyroxine sodium with a complexing agent such as polyvinylpyrrolidone, dissolving the resulting mixture in a polar organic solvent, adding a cellulose carrier such as microcrystalline cellulose, and drying the resulting mixture to yield a complex of levothyroxine sodium adsorbed on the cellulose carrier. However, granulations and dry mixes described therein are disadvantageous in that the drug load is quite low, due to the fact that the granulations and dry mixes contain very large amounts of microcrystalline cellulose, and consequently relatively small amounts of drug.

Solid dispersions primarily have been used to increase bioavailability of drugs. See, for example, Habib, ed. (2001), Pharmaceutical Solid Dispersions, Technomic Publishing Co., Lancaster, Pa.

U.S. Pat. No. 6,197,781 to Guitard et al., incorporated herein by reference, discloses solid dispersions that are said to increase bioavailability of the immunosuppresant rapamycin, which has poor bioavailability. Above-cited U.S. Pat. No. 6,197,781 discloses that the carrier media of such dispersions can comprise hydroxypropylcellulose (HPC), polyvinylpyrrolidone (PVP), cyclodextrin, hydroxypropylmethylcellulose (HPMC), polyethylene glycol (PEG) or polyglycolized glyceride, and can further comprise additional excipients, such as surfactants, flavoring agents, antioxidants, stabilizers and fillers. The fillers mentioned include microcrystalline cellulose and lactose. It should be noted that rapamycin is neither hygroscopic nor deliquescent. Thus, a benefit in reduced moisture sorption is neither disclosed in above-cited U.S. Pat. No. 6,197,781 nor is to be expected with the dispersions disclosed therein.

On the other hand, U.S. Pat. No. 6,204,255 to Klokkers, incorporated herein by reference, discloses non-deliquescent solid dispersions consisting of the hygroscopic and deliquescent drug sodium valproate and cyclodextrin that reportedly do not stick to tablet punches during tableting. However, although the disclosed dispersions, when subjected to humid conditions, absorbed less moisture than unformulated sodium valproate, the dispersions still exhibited 45% moisture absorption at 75% relative humidity. Above-cited U.S. Pat. No. 6,204,255 teaches that excipients such as microcrystalline cellulose can be blended with the formulation after the dispersion has formed. Accordingly, such excipients are not a component of the dispersions themselves.

U.S. Pat. No. 4,223,006 to Taskis, which is incorporated herein by reference, discloses particles consisting of the hygroscopic compound clavulanic acid dispersed in a polymeric binder of low water vapor permeability. Preferred binders are ethylcellulose and polyvinyl acetate phthalate. The particles are said to absorb significantly less moisture when subjected to humid conditions than unformulated clavulanic acid particles. Taskis teaches that the clavulanic acid must be anhydrous and that the process for making the particles of the invention are normally kept as water-free as possible . Above-cited U.S. Pat. No. 4,223,006 teaches that a disintegrant, such as microcrystalline cellulose, can be blended with the particles after the dispersion has formed.

Additionally, hygroscopic and/or deliquescent drugs pose problems that are not directly the result of interactions with humid environments. For example, U.S. Pat. No. 5,037,698 to Brunel, which is incorporated herein by reference, reports that when hygroscopic and/or deliquescent drugs are incorporated into gelatin capsules, a commonly used dosage form, the drugs tend to absorb moisture from the capsules, leaving the capsules in a brittle or deformed state, susceptible to breakage and leakage. Above-cited U.S. Pat. No. 5,037,698 describes a method of “hot filling” a gelatin capsule that is said to address this problem. This method comprises the steps of forming a mixture of a hygroscopic or deliquescent component, such as a drug, with a quantity of water sufficient to prevent embrittlement or softening of the capsule shell, heating the composition to liquid form, adding a thickening agent such as polyethylene glycol, and introducing the resulting suspension or solution into a gelatin capsule. On cooling, the resulting composition is said to attain a solid or semi-solid state. Neither a filler incorporated within the solid or semi-solid composition nor an excipient such as microcrystalline cellulose blended with the composition is specifically contemplated in above-cited U.S. Pat. No. 5,037,698.

Solid dispersions are not usually favored in commercial pharmaceutical formulations, because they pose undue stress on the manufacturing process and often are difficult to incorporate into conventional dosage forms. For example, the hot filled solids and semi-solids described in above-cited U.S. Pat. No. 5,037,698 are disadvantageous, in that hot filling necessarily requires that the capsules be filled immediately upon preparation of the suspension or solution that will become the solid or semi-solid upon cooling. Consequently little room is left, for example, for machine failures or flexibility in manufacturing plant designs and procedures.

Further, solid dispersions comprising drugs and polyethylene glycol are known to have poor handling properties, namely, the dispersions tend to be unpulverizable, sticky masses. See, for example, Habib, ed. (2001), op. cit., p. 81. Such sticky masses are difficult to manufacture, as they have a tendency to clog machinery, and are difficult, if not impossible, to incorporate into dosage forms that are of significant commercial interest, such as tablets and capsules.

Therefore, a need exists for acceptably non-hygroscopic solid dispersions comprising a hygroscopic and/or deliquescent drug that can readily be formulated into convenient dosage forms and that are suitable for large-scale manufacture.

### SUMMARY OF THE INVENTION

It is, therefore, an object of the present invention to provide novel acceptably non-hygroscopic compositions comprising a hygroscopic and/or deliquescent drug.

It is also the object of the present invention to provide acceptably non-hygroscopic compositions of a hygroscopic and/or deliquescent drug in forms that can be easily incorporated into conventional dosage forms, such as tablets and capsules.

Accordingly, there is now provided an acceptably non-hygroscopic pharmaceutical composition comprising a solid dispersion having a hygroscopic and/or deliquescent drug and a carrier medium comprising (a) a matrix forming agent selected from hydroxyethylcellulose, HPC, HPMC, HPMC phthalate, PVP, PEG, polyglycolized glycerides, cyclodextrins and carbomers, and (b) a filler, wherein the drug is dispersed or dissolved in the carrier medium.

The present invention represents a significant advancement over the art cited hereinabove in providing a solid dispersion that need not be in liquid form when transferred to a capsule. Moreover, the solid dispersion of the present invention is unexpectedly more resistant to moisture absorption and has better handling and processing properties than a solid dispersion consisting solely of the drug and polyethylene glycol, or a formulation comprising such a solid dispersion blended with a excipient such as microcrystalline cellulose after the dispersion has formed.

### BRIEF DESCRIPTION OF THE DRAWINGS

FIG. 1 is a graph showing resistance to moisture absorption of a composition of the invention by comparison with unformulated drug.

FIG. 2 is a graph comparing resistance to moisture absorption of a composition of the invention with that of two comparative compositions.

### DETAILED DESCRIPTION OF THE INVENTION

The term “solid dispersion” as used herein means a composite material consisting of an inert carrier medium that is solid at ambient temperature and forms a continuous matrix wherein one or more drugs are homogeneously distributed in solution or in particulate form. Illustratively, a solid dispersion can be prepared by the melting, solvent, or melting-solvent methods, or variations thereof, described in greater detail below and in reference texts, such as Habib, ed. (2001), op. cit. and Chiou & Riegelman (1971), J. Pharm. Sci., 60(9), 1281-1302. The term “solid dispersion” does not include a composition wherein a particulate drug is distributed in one or more particulate solid diluents, prepared for example by traditional mixing. Categories of solid dispersions include, for example, simple eutectic mixtures, solid solutions, glass solutions or suspensions, drug-carrier complexes, amorphous precipitations of drug in a crystalline carrier, etc., as described by Chiou & Riegelman (1971), op. cit.

The term “hygroscopic” as used herein refers to materials, such as drugs or pharmaceutical excipients, that absorb significant amounts of atmospheric moisture when exposed to conditions of normal ambient relative humidity (RH), for example 10-50% RH. The term “deliquescent” refers to drugs or excipients that tend to undergo gradual dissolution and/or liquefaction due to attraction and/or absorption of moisture from air when exposed to these conditions. Those skilled in the art will appreciate that over the usual range of ambient temperatures used in drug formulation, hygroscopicity and the state of deliquescence are largely temperature independent, and that there are varying degrees of hygroscopicity and deliquescence. Thus, for example, adverbs such as “very,” “slightly,” or “extremely” sometimes precede the words “hygroscopic” or “deliquescent” in descriptions of drugs or excipients in order to indicate the amount of moisture a particular drug or excipient tends to absorb in humid climates or the degree to which a particular drug or excipient tends to dissolve and/or liquefy due to attraction and/or absorption of moisture from humid air. As used herein, “hygroscopic” refers to drugs or excipients that are at least slightly hygroscopic. Likewise, “deliquescent” herein refers to drugs or excipients that are at least slightly deliquescent.

The term “acceptably non-hygroscopic pharmaceutical solid dispersion composition” herein refers to a composition that does not absorb substantial amounts of moisture when subjected to relatively humid conditions, for example 40-70% RH. Consequently, shelf life, handling and processing properties of the composition and drug release rate from such a composition are generally not substantially affected by exposure to such conditions. Various methods are known to those skilled in the art for detecting or measuring moisture absorption by a composition; an illustrative method that is convenient and easy to apply in most situations is observation and/or measurement of increase in mass of the composition. Accordingly, a composition of the invention preferably exhibits an increase in mass of less than about 15%, more preferably less than about 10%, and even more preferably less than about 6%, when subjected to conditions of 60% relative humidity and ambient temperatures (21-23° C.) for a time sufficient to achieve substantial equilibrium, i.e., a time after which no further significant increase in mass is observed.

The term “drug” herein refers to one or more agents effective to treat a disease in a subject, wherein “treat” includes identify, prevent, cure, or diagnose.

Illustratively, suitable hygroscopic and/or deliquescent drugs for use in the present invention include, without limitation, drugs from the following classes: abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents (including selective COX-2 inhibitors), antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, antigonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, debriding agents, decongestants, depigmentors, dermatitis herpetiformis suppressants, diagnostic agents, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, a-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H₂ receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, nutraceuticals, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, smoking cessation aids, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, vitamins, xanthine oxidase inhibitors, and combinations thereof.

Non-limiting illustrative examples of hygroscopic and/or deliquescent drugs suitable for use in the present invention include acetylcholine chloride, acetylcamitine, actinobolin, aluminum methionate, aminopentamide, aminopyrine hydrochloride, ammonium bromide, ammonium valerate, amobarbital sodium, anthiolimine, antimony sodium tartrate, antimony sodium thioglycollate, aprobarbital, arginine, aspirin, atropine N-oxide, avoparcin, azithromycin monohydrate, betahistine mesylate, betaine, bethanechol chloride, bismuth subnitrate, bupropion, butamirate, buthalital sodium, butoctamide, cacodylic acid, calcium chloride, calcium glycerophosphate, calcium iodide, carbachol, carnitine, caspofungin, ceruletide, chlorophyllin sodium-copper salt, choline alfoscerate, choline salicylate, choline theophyllinate, cilastatin, citicoline, cobalt dichloride, cromolyn disodium, cupric sulfate pentahydrate, cyanocobalamin, cyclobutyrol, cysteine hydrochloride, deaminooxytocin (L-isomer, anhydrous), deanol hemisuccinate, demecarium bromide, dexamethazone phosphate disodium salt, DL-dexpanthenol, dibucaine hydrochloride, dichlorophenarsine hydrochloride, diclofenac sodium, diethylcarbamazine citrate, dimethyl sulfoxidem, drotebanol, echinomycin, ephedrine (anhydrous), ergotamine, ethanolamine, fencamine hydrochloride, ferric chloride, ferrous iodide, ficin, gadobenate dimeglumine, gentamicin C complex sulfate, guanidine, heparin, hexadimethrine bromide, hexamethonium tartrate, hexobarbital sodium, histamine, hydrastine hydrochloride, hyoscyamine hydrobromide, S-[2-[(1-iminoethyl)amino]ethyl]-2-methyl-L-cysteine, imipramine N-oxide, isometheptene hydrochloride, isosorbide, levothyroxine sodium, lichenifonnins, lobeline sulfate, magnesium chloride hexahydrate, magnesium trisilicate, menadione, mercaptomerin sodium, mersalyl, metaraminol, methacholine chloride, methantheline bromide, methantheline chloride, methitural sodium, L-methyldopa sesquihydrate, methylmethioninesulfonium chloride, mildiomycin, minocycline hydrochloride, mitoxantrone dihydrochloride, morpholine, muscarine chloride, nafronyl acid oxalate, narceine, nicotine, nicotinyl alcohol, nolatrexed dihydrochloride, omeprazole, oryzacidin, oxalic acid, oxophenarsine hydrochloride, panthenol, pantothenic acid (sodium salt), papain, penicillamine hydrochloride, penicillin G (potassium salt), pentamethonium bromide, pentamidine isethionate, pepsin, perazine dihydrochloride, phenobarbital, sodium 5,5-diphenyl hydantoinate, phethenylate sodium, phosphocreatine (calcium salt tetrahydrate), physostigmine sulfate, pilocarpine hydrochloride, pipemidic acid, podophyllotoxin-β-D-glucoside, potassium carbonate, potassium iodide, pralidoxime mesylate, prednisolone sodium phosphate, procainamide hydrochloride, procaine butyrate, L-proline, promazine hydrochloride, propamidine isethionate, prostacyclin sodium, pyridostigmine bromide, pyronaridine, quinacillin disodium, quinoline, radioactive sodium iodide, reserpilic acid dimethylaminoethyl ester dihydrochloride, secobarbital sodium, silver fluoride, sodium acetate, sodium bromide, sodium propionate, sodium dibunate, sodium dichromate(VI), sodium nitrite, sodium pentosan polysulfate, sodium valproate, soluble sulfamerazine, stibocaptate, streptomycin, succinylcholine bromide, succinylcholine iodide, sulfaquinoxaline, sulisatin disodium, suramin sodium, tamoxifen citrate, taurocholic acid, terazosin hydrochloride, thiobutabarbital sodium, thiopental sodium, ticarcillin disodium, 2,2,2-trichloroethanol, trientine, triethanolamine, triftazin, tolazoline hydrochloride, vinbarbital sodium, viomycin, vitamin B₁₂, zinc iodide, and combinations, pharmaceutically acceptable hygroscopic and/or deliquescent salts and variants thereof.

Preferred drugs include acetylcholine chloride, actinobolin, aminopentamide, aminopyrine hydrochloride, ammonium valerate, atropine N-oxide, avoparcin, betaine, bupropion, calcium chloride, calcium iodide, carnitine, choline alfoscerate, choline salicylate, deaminooxytocin (L-isomer, anhydrous), dimethyl sulfoxidem, ergotamine, ferric chloride, ferrous iodide, guanidine, hexobarbital sodium, hyoscyamine hydrobromide, S-[2-[(1-iminoethyl)amino]ethyl]-2-methyl-L-cysteine, imipramine N-oxide, isometheptene hydrochloride, magnesium chloride hexahydrate, methantheline chloride, methitural sodium, methylmethioninesulfonium chloride, muscarine chloride, narceine, nicotine, nicotinyl alcohol, physostigmine sulfate, potassium iodide, pralidoxime mesylate, quinacillin disodium, silver fluoride, sodium propionate, sodium dichromate (VI), sodium valproate, streptomycin, taurocholic acid, triethanolamine, and hygroscopic and/or deliquescent salts thereof.

In view of the superior moisture protection qualities afforded by the dispersions described herein, the present invention is particularly advantageous where the drug selected for use in such a dispersion is deliquescent and/or has a hygroscopicity such that when unformulated the drug exhibits at least about 15% mass increase at equilibrium when exposed to 60% relative humidity at ambient temperature.

In a preferred embodiment, the drug is nicotine. Nicotine is useful in pharmaceutical formulations as, for example, an aid in smoking cessation.

In another preferred embodiment, S-[2-[(1-iminoethyl)amino]ethyl]-2-methyl-L-cysteine is the drug used in a composition of the invention. This drug, disclosed in International Patent Publication No. WO 01/72703, incorporated herein by reference, is a nitric oxide synthase (NOS) inhibitor, and is believed to have value in, for example, treating inflammation and other NOS-mediated disorders, such as pain, headache and fever. S-[2-[(1-iminoethyl)amino]ethyl]-2-methyl-L-cysteine for use herein can be prepared by any suitable means, including processes described in above-cited International Patent Publication No. WO 01/72703. This compound can be used in its free base form or as a pharmaceutically acceptable salt, for example the dihydrochloride salt.

It has now been found that S-[2-[(1-iminoethyl)amino]ethyl]-2-methyl-L-cysteine and its dihydrochloride salt are extremely hygroscopic and deliquescent. It is particularly surprising that such a hygroscopic and deliquescent drug can be formulated in accordance with the present invention as an acceptably non-hygroscopic composition.

The drug is preferably present in an amount of at least about 5%, more preferably at least about 10%, by weight of the composition. Indeed, the present inventors have observed that a solid dispersion as provided herein affords acceptable protection from moisture absorption even where the composition contains as much as 60% by weight of S-[2-[(1-iminoethyl)amino]ethyl]-2-methyl-L-cysteine. A practical upper limit of drug concentration in a composition of the invention depends on, for example, the amount of moisture absorption that can be tolerated and the degree of hygroscopicity and/or deliquescence of the drug, it being contemplated that less hygroscopic and/or deliquescent drugs will require lesser amounts of carrier medium than drugs that are more hygroscopic and/or deliquescent, and that where the amount of carrier medium is lower the drug concentration can be higher.

Accordingly, compositions of the invention comprise about 1% to about 75%, preferably about 5% to about 65%, and more preferably about 10% to about 60% of a hygroscopic and/or deliquescent drug by weight of the composition (i.e. weight of the drug per weight of the composition).

The term “matrix forming agent” herein refers to a polymer that itself or in combination with a filler and/or any other excipient or excipients, is able to create a matrix wherein the hygroscopic and/or deliquescent drug can be dispersed or dissolved.

In one embodiment, the matrix forming agent is HPC. Exemplary HPCs useful in the present invention include those having low dynamic viscosity in aqueous media, preferably below about 400 cps, e.g., below about 150 cps as measured in a 2% aqueous solution at 25° C. Preferred HPCs have a low degree of substitution, and an average molecular weight below about 200,000 daltons, e.g., from about 50,000 to about 150,000 daltons. HPC is commercially available, for example, under the trade names Klucel™ LF, Klucel™ EF and Klucel™ JF (Aqualon), and Nisso™ HPC-L (Nippon Soda).

In another embodiment, the matrix forming agent is a cyclodextrin, for example a β-cyclodextrin or an α-cyclodextrin. Examples of suitable β-cyclodextrins include methyl-β-cyclodextrin, dimethyl-β-cyclodextrin, hydroxypropyl-β-cyclodextrin (HPBCD), glycosyl-β-cyclodextrin, maltosyl-β-cyclodextrin, sulfo-β-cyclodextrin and sulfo-alkylethers, e.g., sulfo-C₁₋₄-alkylethers, of β-cyclodextrin. Examples of α-cyclodextrins include glucosyl-α-cyclodextrin and maltosyl-α-cyclodextrin. β-Cyclodextrins such as HPBCD are especially preferred for use in the present invention.

In another embodiment, the matrix forming agent is a polyglycolized glyceride. Polyglycolized glycerides are generally mixtures of monoesters, diesters and triesters of glycerol with monoesters and diesters of polyethylene glycols having a average molecular weight of about 200 and 6000. They can be obtained by partial transesterification of triglycerides with polyethylene glycol or by esterification of glycerol and polyethylene glycol with fatty acids using known reactions. Preferably, such fatty acids have 8-22, more preferably 8-18, carbon atoms. Examples of natural vegetable oils, which may be used as a source of such fatty acids, include palm kernel oil and palm oil. The polyethylene glycol can optionally be replaced with another polyol, for example a polyglycerol or sorbitol. Polyglycolized glycerides are available for example under the trade name Gelucire® (Gattefosse).

In another embodiment, the matrix forming agent is hydroxyethylcellulose. Exemplary hydroxyethylcelluloses useful in the invention include those having low dynamic viscosity in aqueous media, preferably below about 400 cps, e.g., below about 150 cps as measured in a 2% aqueous solution at 25° C. Hydroxyethylcellulose is available for example under the trade names Cellosize™ (Amerchol) and Natrusol™ (Aqualon).

In another embodiment, the matrix forming agent is HPMC phthalate, which is available for example from Shin-Etsu.

In a preferred embodiment, the matrix forming agent is a carbomer. Carbomers are high molecular weight polymers of acrylic acid that are cross-linked with either allylsucrose or allyl esters of pentaerythritol. Carbomers are available, for example, under the trade name Carbol™ (Noveon Pharmaceuticals).

In another preferred embodiment, the matrix forming agent is HPMC. Good results can be obtained using HPMC with a low apparent dynamic viscosity, preferably below about 100 cps as measured at 20° C. for a 2% by weight aqueous solution, more preferably below about 50 cps, most preferably below about 20 cps, for example 3 cps. HPMC, including a grade having apparent dynamic viscosity of 3 cps, is available for example under the trade name Pharmacoat™ 603 (Shin-Etsu).

In yet another preferred embodiment, the matrix forming agent is PVP, also known as povidone. PVP is available for example under the trade names Plasdone™ (ISP) and Kollidon™ (BASF). PVP having an average molecular weight of about 8,000 to about 50,000 daltons is preferred.

In an especially preferred embodiment, the matrix forming agent is a PEG that is solid at ambient temperatures. Such PEGs include those that have an average molecular weight of about 1,000 daltons to about 35,000 daltons, for example about 8,000 daltons. PEG is available for example under the trade name Carbowax™ (Dow).

Compositions comprising combinations and/or variants of one or more of the above-described matrix forming agents are also encompassed by the present invention.

The matrix forming agent is present in an amount of about 10% to about 95%, preferably about 20% to about 85%, more preferably about 25% to about 75% by weight of the composition.

The term “filler” herein refers to inert materials that serve to increase the mass and/or bulk density of the solid dispersion, so that, for example, the solid dispersion can be relatively easily incorporated into a conventional dosage form, e.g., a tablet or capsule. Fillers contemplated for use in the present invention include for example microcrystalline cellulose, lactose, calcium carbonate, carboxymethylcellulose calcium, carboxymethylcellulose sodium, dibasic calcium phosphate dihydrate, tribasic calcium phosphate, calcium sulfate, dextrose, ethyl cellulose, fructose, kaolin, magnesium carbonate, magnesium stearate, magnesium trisilicate, maltol, maltodextrin, mannitol, methyl cellulose, powdered cellulose, pregelatinized starch, starch, sterilizable maize starch, compressible sugar, confectioner's sugar and the like. Preferably the filler used does not adversely affect the stability and/or dissolution performance of the dispersion.

In a particularly surprising finding, a composition of the invention having a filler that is itself hygroscopic and/or deliquescent exhibits remarkably low hygroscopicity and can provide a free-flowing solid. In this regard, it is to be noted that when a hygroscopic and/or deliquescent filler is blended with a solid dispersion after the dispersion matrix has formed, as described in above-cited U.S. Pat. No. 6,204,255, the filler is not protected from moisture absorption. In a composition prepared by simple blending of a hygroscopic and/or deliquescent filler with a solid dispersion, such moisture absorption can lead to an increase in mass of the composition when the composition is exposed to high humidity. However, as demonstrated herein, a composition of the present invention, even one using a hygroscopic and/or deliquescent filler, exhibits much reduced tendency for moisture absorption and represents a significant advance in the art.

Such hygroscopic and/or deliquescent fillers include for example microcrystalline cellulose, tribasic calcium phosphate, anhydrous calcium sulfate, carboxymethylcellulose calcium, carboxymethylcellulose sodium, anhydrous dextrose, fructose, anhydrous lactose, anhydrous magnesium stearate, magnesium trisilicate, maltodextrin, methylcellulose, powdered cellulose, pregelatinized starch, starch, sterilizable maize starch, compressible sugar, confectioner's sugar and the like.

Preferably the filler is a hygroscopic and/or deliquescent cellulosic polymer, e.g., microcrystalline cellulose, carboxymethylcellulose sodium, carboxymethylcellulose calcium, methylcellulose or powdered cellulose.

Most preferably the filler is microcrystalline cellulose, available for example under the trade name Avicel™ (FMC) in various grades.

Preferably the filler is present in an amount sufficient to enable the solid dispersion, once formed, to be in a flowable state, such as a powder, that can be easily incorporated into conventional dosage forms, such as tablets and capsules. Accordingly, the filler is generally present in an amount of about 1% to about 95%, preferably about 5% to about 30% by weight of the composition. The present inventors have found that hygroscopic and/or deliquescent cellulosic polymers, such as microcrystalline cellulose, in an amount of about 20% by weight of the composition are particularly well-suited for the present invention, as in combination with a hygroscopic and/or deliquescent drug and a matrix forming agent as described above, such cellulosic polymers surprisingly allow the solid dispersion to be easily incorporated into conventional dosage forms.

If desired, the carrier medium can further comprise other pharmaceutically acceptable excipients selected, for example, from antioxidants such as α-tocopherol, ascorbic acid, ascorbyl palmitate, butylated hydroxyanisole and butylated hydroxytoluene; disintegrants such as sodium starch glycolate and sodium starch fumarate; flavoring agents such as aspartame, saccharin and saccharin sodium; glidants such as magnesium aluminum silicate, talc and titanium dioxide; lubricants such as stearic acid; neutralizing agents such as dibasic sodium phosphate and monobasic sodium phosphate; preservatives; stabilizers; surfactants such as docusate sodium and sorbitan esters; wetting agents such as poloxamers and sodium lauryl sulfate; and thickeners and coatings such as gelatin and polymethacrylates. Such excipients can alternatively or additionally be later blended with the solid dispersion, once it has formed, prior or subsequent to incorporation into a pharmaceutical dosage form.

In addition, if a controlled release formulation is desired, the carrier medium can further comprise a wax, for example cetyl esters, anionic or nonionic emulsifying wax, carnauba wax, microcrystalline wax or the like, or the dispersion or a dosage form comprising the dispersion can be coated with one or more polymers commonly used in controlled release formulations, such as polymethacrylates available for example as Eudragit™ of Rohm. It is also contemplated that controlled release formulations can be achieved by using viscous grades of the matrix forming agents or high molecular weight polyethylene glycols.

Alternatively, the composition can be in the form of an immediate release formulation having a decreased time of onset of therapeutic effect. In preparing an immediate release composition of the invention, excipients such as disintegrants can if desired be added to the carrier medium, or to the dispersion prior or subsequent to incorporation into a dosage form.

If additional moisture protection is desired, for example because the drug is extremely hygroscopic and/or deliquescent or very little moisture uptake can be tolerated, a dispersion of the invention can be coated with one or more polymers, such as ethylcellulose, HPC or HPMC.

If desired, a solid dispersion of the invention, or a composition containing such solid dispersion, can comprise, in addition to the hygroscopic and/or deliquescent drug, a non-hygroscopic, non-deliquescent drug. However, preferably the dispersion or composition is substantially free of such non-hygroscopic, non-deliquescent drugs.

Dispersions of the invention can be prepared by any suitable process. Known methods of preparing solid dispersions include solvent, fusion, or fusion-solvent methods as described in standard reference texts, such as Habib (2001), op. cit., pp. 20-26. The processes described below are presented for illustrative purposes, and are not intended to limit the scope of the invention.

In one embodiment, a solid dispersion is prepared according to the solvent method, by dissolving a matrix forming agent, a filler and a hygroscopic and/or deliquescent drug in a solvent. Solvents contemplated for use in this process include water; alcohols such as methanol, ethanol and isopropanol; esters such as ethyl acetate; ethers such as diethyl ether; ketones such as acetone; halogenated hydrocarbons such as dichloroethane; and combinations thereof such as a mixture of ethanol and acetone. The solvent is then evaporated, for example using elevated temperature and/or a vacuum, or by freeze drying or spray drying. As the solvent evaporates, supersaturation occurs, followed by simultaneous precipitation of both the matrix forming agent and the drug in solid form. The resulting precipitate, which has the drug dissolved or suspended in a carrier medium formed from the matrix forming agent and the filler, is then dried to produce a solid dispersion of the invention. This process is especially useful for drugs that are soluble in the carrier medium selected and for drugs that are thermolabile.

In another embodiment, a solid dispersion is prepared according to the fusion method, wherein a matrix forming agent is heated to a temperature above its melting point and a hygroscopic and/or deliquescent drug is added with mixing to the melted agent. A filler is either heated along with the matrix forming agent or incorporated along with the drug by mixing after the melting of the matrix forming agent. The resulting composition is then cooled, for example allowed to cool naturally, with constant mixing, e.g., by stirring, to produce a formulation that is a solid dispersion having the drug evenly dispersed therein. If the drug is soluble in the matrix forming agent, it remains dissolved in the formulation, which is therefore a solid solution or molecular dispersion. If the drug is not soluble in the matrix forming agent, it is dispersed in crystalline or amorphous particulate form in the solid dispersion.

In yet another embodiment, a solid dispersion is prepared according to the fusion-solvent method, wherein a matrix forming agent is heated until melted and a solution of a hygroscopic and/or deliquescent drug in a suitable solvent is added with mixing thereto. Again, a filler is either heated along with the matrix forming agent or is incorporated along with the drug by mixing after the melting of the matrix forming agent. If, upon cooling, the resulting composition is capable of holding a certain proportion of solvent while maintaining its solid properties, and if the solvent is innocuous, the need for solvent removal is eliminated; otherwise, the solvent is removed, for example using elevated temperature and/or a vacuum, or by freeze drying or spray drying.

According to the fusion and fusion-solvent methods, it is preferred to heat to a temperature only sufficiently high to result in melting of the matrix forming agent, to avoid unnecessary exposure of the drug to excessive heat and consequent risk of thermal degradation or other adverse effect.

Selection of a method of preparing a solid dispersion will be influenced by various factors including solubility of the drug in the carrier medium, as well as the advantages and disadvantages associated with each method of preparation.

Preferably, in the case of S-[2-[(1-iminoethyl)amino]ethyl]-2-methyl-L-cysteine, which has low solubility in HPMC and PEG, a dispersion is prepared by the fusion method or the fusion-solvent method, particularly if HPMC or PEG is selected as a matrix forming agent.

Compositions of the invention are useful for administration to a subject in order to treat, identify, prevent or cure a disease in the subject. Administration can be by any suitable route, including without limitation oral, buccal, sublingual, topical and rectal routes.

In one embodiment, a composition is provided in a dosage form suitable for rectal administration, for example as a suppository. Preferably, however, the composition is provided in a dosage form suitable for oral administration. The term “oral administration” herein includes any form of delivery of a therapeutic agent or a composition thereof to a subject wherein the agent or composition is placed in the mouth of the subject, whether or not the agent or composition is immediately swallowed. Thus “oral administration” includes buccal and sublingual as well as esophageal (peroral) administration. Absorption of the agent can occur in any part or parts of the gastrointestinal tract including the mouth, esophagus, stomach, duodenum, ileum and colon. The term “orally deliverable” herein means suitable for oral administration.

Most preferably, the composition is provided in the form of a tablet. A tablet of the invention can be of any suitable color, texture, type (e.g., effervescent, non-effervescent, sublingual, etc.) and shape (e.g., round, oval, biconcave, hemispherical, square, rectangular, polygonal, etc.). The tablet preferably has a total weight of about 10 mg to about 1000 mg, more preferably about 20 mg to about 500 mg.

Dosage forms incorporating a dispersion of the present invention can be prepared by any suitable means. For example, the dispersion can be incorporated into capsules, in accordance with the “hot filling” method described in above-cited U.S. Pat. No. 5,037,698, wherein the liquid that will form into a solid dispersion is transferred to a capsule and becomes a solid dispersion in the capsule upon cooling. However, in view of the above-mentioned disadvantages associated with hot filling, it is preferred that the solid dispersion itself, not the liquid form of the dispersion prior to cooling, is incorporated into a dosage form.

In one preferred embodiment, the solid dispersion is sieved and milled. The milled dispersion, optionally combined with excipients, can then be compressed or molded to form tablets, filled into sachets or hard or soft capsules (e.g., gelatin or HPMC capsules) or incorporated into any other desired dosage form.

### EXAMPLES

The following examples are presented to further illustrate the invention. The invention is illustrated with particular reference to S-[2-[(1-iminoethyl)amino]ethyl]-2-methyl-L-cysteine dihydrochoride, herein identified as “Compound A”.

Moisture Uptake Analysis

The dynamic vapor sorption (DVS) approach was used to determine the amount by which the mass of a composition increased upon exposure to various predetermined relative humidities.

### Example 1

### Solid Dispersion of the Invention

A solid dispersion having the ingredients shown in Table 1 was prepared by the method described below.

TABLE 1
Composition of solid dispersion of Example 1

| Ingredient | Quantity (mg) |
|---|---|
| Compound A, lyophilized powder | 25.53 |
| Carbowax ™ 8000¹ | 75.15 |
| Avicel ™ PH-101² | 25.60 |

¹PEG 8000
²microcrystalline cellulose

A capped 7 ml glass vial containing the PEG 8000 was placed in a 68° C. water bath and stirred, with the aid of a small magnetic stirrer, at a low rotation speed until the PEG 8000 had melted. Lyophilized Compound A was placed in a separate 2 ml glass vial to which 1.0 ml methanol was then added. The 2 ml vial was then capped and sonicated for 5 minutes to obtain a clear solution.

This solution was then added to the PEG 8000 in the 7 ml vial under constant stirring in the 68° C. water bath for 5 minutes. Then, with continuing stirring, the microcrystalline cellulose was added. The vial was uncapped and the resulting mixture stirred vigorously for an additional 5 minutes. The vial was then removed from the water bath and cooled, under constant stirring, to ambient conditions. The vial was then placed overnight in a 40° C. vacuum oven. Finally, the vial was removed from the oven and the resulting solid dispersion was gently dislodged with a metal spatula. The solid dispersion was in the form of a free-flowing white powder suitable for tableting.

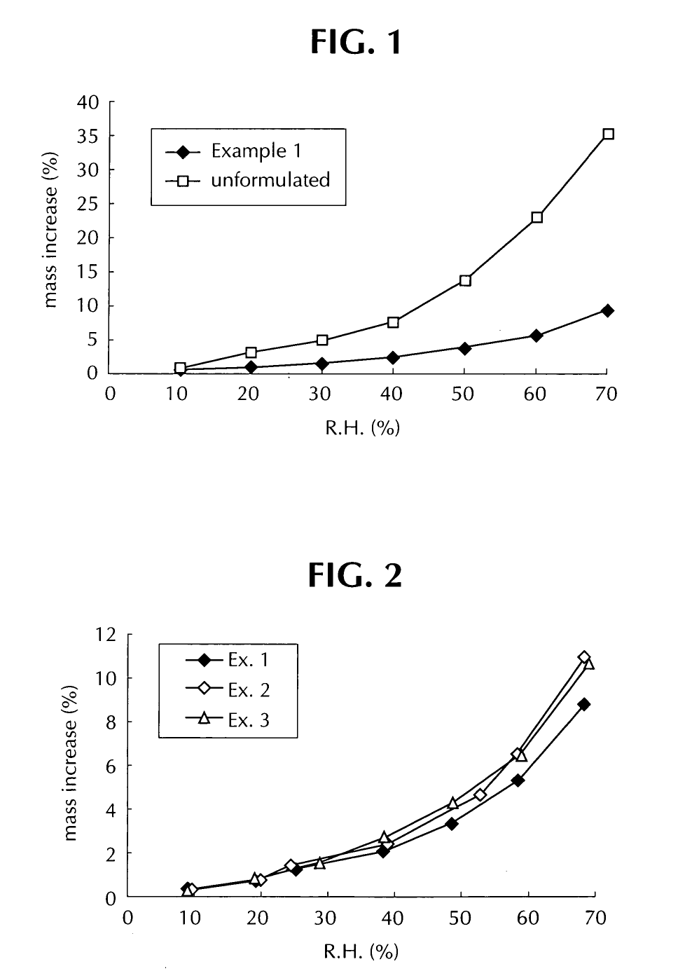

In a moisture uptake analysis, it was found that the solid dispersion of this example exhibited greatly improved resistance to moisture uptake by comparison with unformulated, amorphous Compound A, especially at high relative humidities, as shown in FIG. 1. Upon storage at 40% relative humidity for 100 hours, the solid dispersion remained free flowing.

A scanning electron microscope (SEM) study with sulfur mapping revealed very homogeneous distribution of sulfur in particles of the powder produced in this example. As sulfur occurs in Compound A but not in the excipients used, this result shows very uniform distribution of drug in the particles.

In physical and chemical stability studies carried out on the solid dispersion of this example for about 24 weeks at 40° C. and 75% relative humidity, good drug and formulation stability was observed. X-ray powder diffraction (XRPD) studies showed no change in crystalline form of the drug under the same storage conditions.

### Example 2

### Comparative Solid Dispersion

A solid dispersion having the ingredients shown in Table 2 was prepared by the method described below. It will be noted that the solid dispersion of this example differs from that of Example 1 in lacking a filler (microcrystalline cellulose).

TABLE 2
Composition of solid dispersion of Example 2

| Ingredient | Quantity (mg) |
|---|---|
| Compound A, lyophilized powder | 26.11 |
| Carbowax ™ 8000 | 75.98 |
| Avicel ™ PH-101 | 0 |

A capped 7 ml glass vial containing the PEG 8000 was placed in a 68° C. water bath and stirred, with the aid of a small magnetic stirrer, at a low rotation speed until the PEG 8000 had melted. Lyophilized Compound A was placed in a separate 2 ml glass vial to which 1.0 ml methanol was then added. The 2 ml vial was then capped and sonicated for 5 minutes to obtain a clear solution.

This solution was then added to the PEG 8000 in the 7 ml vial under constant stirring in the 68° C. water bath for 5 minutes. The vial was then removed from the water bath and cooled, under constant stirring, to ambient conditions. The vial was then placed overnight in a 40° C. vacuum oven. Finally, the vial was removed from the oven and the resulting solid dispersion was gently dislodged with a metal spatula. The solid dispersion was in the form of a waxy mass not readily suitable for tableting.

### Example 3

### Comparative Composition

A composition having the ingredients shown in Table 3 was prepared by the method described below. It will be noted that the composition of this example differs from that of Example 1 in that the filler (microcrystalline cellulose) was blended with the solid dispersion after preparation of the solid dispersion.

TABLE 3
Composition of Example 3

| Ingredient | Quantity (mg) |
|---|---|
| Solid dispersion of Example 2 | 48.12 |
| Avicel ™ PH-101 | 12.27 |

The product of Example 2 above was transferred to a clean 7 ml glass vial. The microcrystalline cellulose was added to the vial, which was then capped and mixed using a tubular mixer for 24 hours. The resulting composite was in the form of a sticky substance not readily suitable for tableting.

### Comparison of Moisture Absorption by Compositions of Examples 1-3

As shown in FIG. 2, resistance to moisture absorption of the composition of Example 1 of the invention was superior to that of the comparative compositions of Examples 2 and 3.

Table 4 below further illustrates the superiority of the composition of the invention in resisting moisture absorption. Table 4 shows the mass increase of each composition of Examples 1-3 following exposure to different relative humidities. Also shown in Table 4 for each of the comparative compositions (Examples 2 and 3) are data for relative mass increase, calculated by dividing the mass increase of the comparative composition by that of the composition of Example 1 of the invention at similar relative humidity.

For example, at 38-39% R.H., the comparative composition of Example 2 exhibited 18% greater, and that of Example 3 32% greater, moisture absorption than the composition of the invention. The finding that the composition of the invention absorbed significantly less moisture than the comparative composition of Example 2 is especially surprising, as these compositions differ only in the presence of filler. This result suggests that the filler itself is able to impart improved moisture resistance.

TABLE 4
Mass increase of compositions of Examples 1-3

| Example 1 | | Example 2 | | | Example 3 | | |
|---|---|---|---|---|---|---|---|
| (invention) | | (comparative) | | | (comparative) | | |
| | Mass | | Mass | Relative | | Mass | Relative |
| % | increase | % | increase | mass | % | increase | mass |
| R.H. | (%) | R.H. | (%) | increase¹ | R.H. | (%) | increase¹ |
| 9.0 | 0.28 | 9.8 | 0.26 | 0.93 | 9.3 | 0.29 | 1.04 |
| 19.3 | 0.67 | 20.0 | 0.71 | 1.06 | 19.4 | 0.77 | 1.15 |
| 25.1 | 1.21 | 24.5 | 1.37 | 1.13 | 28.9 | 1.52 | 1.26 |
| 38.4 | 2.02 | 39.1 | 2.39 | 1.18 | 38.6 | 2.67 | 1.32 |
| 48.6 | 3.24 | 52.9 | 4.57 | 1.41 | 48.8 | 4.25 | 1.31 |
| 58.4 | 5.24 | 58.4 | 6.48 | 1.24 | 58.7 | 6.41 | 1.22 |
| 68.3 | 8.72 | 68.3 | 10.9 | 1.25 | 68.9 | 10.59 | 1.21 |

¹Mass increase relative to that of Example 1

1. A pharmaceutical composition comprising a drug and a carrier medium wherein said carrier medium comprises (a) a matrix forming agent selected from the group consisting of hydroxyethylcelluloses, hydroxypropylcelluloses, hydroxypropylmethylcelluloses, hydroxypropylmethylcellulose phthalates, polyvinylpyrrolidones, polyethylene glycols, polyglycolized glycerides, cyclodextrins, carbomers and combinations thereof, and (b) a filler;
wherein the drug is hygroscopic and/or deliquescent and is dispersed in the carrier medium, and wherein the composition is a solid dispersion and is acceptably non-hygroscopic.

2. The composition of claim 1 wherein the drug has a hygroscopicity such that when unformulated the drug exhibits at least about a 15% mass increase when exposed to 60% relative humidity at 21-23° C. for a period of 24 hours or for a period of time sufficient for the composition to reach equilibrium.

3. The composition of claim 1 wherein the drug is a NOS inhibitor, nicotine, or S-[2-[(1-iminoethyl)amino]ethyl]-2-methyl-L-cysteine, or a pharmaceutically acceptable salt thereof.

4. The composition of claim 1 wherein the drug is present in an amount of about 1% to about 75% by weight of the composition.

5. The composition of claim 1 wherein the filler is hygroscopic and/or deliquescent.

6. The composition of claim 1 wherein the filler is present in an amount sufficient to provide a flowable solid dispersion.

7. The composition of claim 1 wherein the filler is selected from the group consisting of tribasic calcium phosphates, anhydrous calcium sulfates, carboxymethylcellulose calciums, carboxymethylcellulose sodiums, anhydrous dextroses, fructoses, anhydrous lactoses, anhydrous magnesium stearates, magnesium trisilicates, maltodextrins, methylcelluloses, microcrystalline celluloses, powdered celluloses, pregelatinized starchs, starchs, sterilizable maize starchs, compressible sugars, confectioner's sugars and combinations thereof.

8. The composition of claim 1 wherein the filler is a microcrystalline cellulose.

9. The composition of claim 1 wherein the filler is present in an amount of about 1% to about 95% by weight of the composition.

10. The composition of claim 1 wherein the matrix forming agent is present in an amount of about 10% to about 95% by weight of the composition.

11. The composition of claim 1 wherein the polyethylene glycol, if present, has an average molecular weight of about 1,000 to about 35,000 daltons.

12. The composition of claim 1 wherein the composition is in a form suitable for oral administration.

13. The composition of claim 1 wherein the composition is in the form of a tablet.

14. The composition of claim 1 wherein the composition exhibits a mass increase of less than about 10% when exposed to 60% relative humidity at 21-23° C. for a period of 24 hours or for a period of time sufficient for the composition to reach equilibrium.

15. The composition of claim 1 wherein the composition is formed by a method selected from the group consisting of a solvent method, a fusion method, and a fusion-solvent method.

16. A pharmaceutical composition comprising (1) a drug and (2) a carrier medium that comprises (a) polyethylene glycol, with an average molecular weight of about 8,000 daltons, present in an amount of about 10% to about 95%, by weight of the composition, and (b) microcrystalline cellulose present in an amount of about 1% to about 95%, by weight of the composition wherein:
i. the drug is S-[2-[(1-iminoethyl)amino]ethyl]-2-methyl-L-cysteine, or a pharmaceutically acceptable salt thereof, and present in an amount of about 1% to about 75% by weight of the composition, and dispersed in the carrier medium, and
ii. the composition exhibits a mass increase of less than about 15% when exposed to 60% relative humidity at 21-23° C. for a period of 24 hours or for a period of time sufficient for the composition to reach equilibrium.

17. A process for making a composition of claim 1, the process comprising:
(i) dissolving in a solvent, in any order or simultaneously, (a) a hygroscopic and/or deliquescent drug, (b) a filler and (c) a matrix forming agent selected from the group consisting of hydroxypropylcelluloses, hydroxypropylmethycelluloses, hydroxypropylmethylcellulose phthalates, polyvinylpyrrolidones, polyethylene glycols, polyglycolized glycerides, cyclodextrins and combinations thereof; and
(ii) removing the solvent using elevated temperature or a vacuum, or by freeze drying or spray drying to form a solid dispersion of the drug in a carrier medium that comprises the filler and the matrix forming agent.

18. A process for making a composition of claim 1, the process comprising:
(i) heating a matrix forming agent selected from the group consisting of hydroxypropylcelluloses, hydroxypropylmethycelluloses, hydroxypropylmethylcellulose phthalates, polyvinylpyrrolidones, polyethylene glycols, polyglycolized glyceridess, cyclodextrins and combinations thereof to a temperature above its melting point;
(ii) adding, in any order or simultaneously, to the resulting melted matrix forming agent a filler and a hygroscopic and/or deliquescent drug with mixing to form a composite; and
(iii) cooling the composite with mixing to form a solid dispersion of the drug in a carrier medium that comprises the filler and the matrix forming agent.
